(19) United States
(12) Patent Application Publication
(10) Pub. No.: US 2012/0237958 A1
(43) Pub. Date: Sep. 20, 2012

(54) METHODS FOR IDENTIFYING ANIMALS WITH A REDUCED ABILITY TO DIGEST LIPID AND PROTEIN

(76) Inventors: Carolyn Jean Cupp, Liberty, MO (US); Avinash Patil, Hillsboro, OR (US)

(21) Appl. No.: 13/499,162

(22) PCT Filed: Oct. 8, 2010
(86) PCT No.: PCT/US10/02724

§ 371 Date: Jun. 5, 2012

Related U.S. Application Data
(60) Provisional application No. 61/279,044, filed on Oct. 15, 2009.

(51) Int. Cl. G01N 33/53; A61B 5/107; G09B 19/00; A61B 5/103

(52) U.S. Cl. 435/7.92; 600/587; 434/219

(57) ABSTRACT

The invention provides methods for identifying animals with a reduced ability to digest fat or protein by determining various blood and physical factors for the animals and comparing the determined values to standard values that are indicative of a reduced ability to digest fat and protein.

### CROSS REFERENCE TO RELATED APPLICATIONS

This application is a national stage application under 35 U.S.C. §371 of PCT/US2010/002724 filed Oct. 8, 2010, which claims priority to U.S. Provisional Application Ser. No. 61/279,044 filed Oct. 15, 2009, the disclosures of which are incorporated herein by this reference.

### BACKGROUND OF THE INVENTION

1. Field of the Invention

The invention relates generally to methods for identifying animals with a reduced ability to digest food nutrients and particularly to methods for identifying animals with a reduced ability to digest lipid or protein.

2. Description of Related Art

An animal's ability to digest foods and food nutrients is known to change with age. Similarly, methods for assessing an animal's ability to digest foods and food nutrients and methods for affecting digestibility are known, e.g., “Changing Perspectives on Aging and Energy Requirements Aging and Digestive Function in Humans, Dogs and Cats,” The Journal of Nutrition Vol. 128 No. 12 Dec. 1998, pp. 2632S-2635S and Experimental Approaches to Study the Nutritional Value of Food Ingredients for Dogs and Cats,” R. Bras. Zootec. vol. 36 supp 1.0 Viçosa July 2007. US20050147649 discloses methods and dietary compositions for improving fat digestibility by animals. US20090136588 discloses methods for altering food intake, modifying nutrient digestibility and altering stool quality and/or stool frequency, e.g., methods for increasing or decreasing protein digestibility. WO03084344 and WO03084343 disclose methods for providing animals with benefits relating to the effective assimilation of lipid or lipid fractions by administering edible compositions containing ingredients that maintain, promote, or enhance the pet's capacity to digest lipid efficiently. These methods, while useful for testing digestibility, often require lengthy and complicated procedures that are difficult or inconvenient, particularly in clinical situations. There is, therefore, a need for new methods for identifying animals with a reduced ability to digest lipid and protein.

### SUMMARY OF THE INVENTION

It is, therefore, an object of the invention to provide methods for identifying animals with a reduced ability to digest lipid.

It is another object of the invention to provide methods for identifying animals with a reduced ability to digest protein.

These and other objects are achieved by determining for an animal at least two of the factors selected from the group consisting of Vitamin E concentration; Vitamin B12 (also known as cobalamin) concentration; folate concentration; pancreatic lipase immunoreactivity; body condition score; skin fold thickness; and age and, for the corresponding factors, determining that the animal has a reduced ability to digest either lipid or protein, or both, if Vitamin E concentration is less than or equal to 5 mg/L; Vitamin B12 concentration is less than or equal to 200 ng/L; folate concentration is less than or equal to 15 μg/L; pancreatic lipase immunoreactivity is greater than or equal to 3.5 μg/L; body condition score is less than 3 on a 5 point scale; skin fold thickness is less than or equal to 2 mm; and age is equal to or greater than 70% of the average lifespan for the type of animal. As described herein, various factors are used for identifying animals with a reduced ability to digest fat and various factors are useful for identifying animals with a reduced ability to digest protein.

Additional and further objects, features, and advantages of the invention will be readily apparent to those skilled in the art.

### DETAILED DESCRIPTION OF THE INVENTION

### Definitions

Abbreviations: Nanograms (ng); liters (L), micrograms (μg); milligrams (mm); and milligrams (mg).

The terms “Vitamin E concentration”; Vitamin B12 concentration”; “folate concentration”; and “pancreatic lipase immunoreactivity” mean the concentration or activity of the respective compounds in the blood of an animal as determined by any suitable method.

The term “digest” means the processes of digestion, absorption, and assimilation. The digestion process converts food into absorbable nutrients in the gastrointestinal tract using mechanical and chemical processes that break the food into nutrients that can be absorbed. Absorption is the process that facilitates the passage of digested food nutrients from the gastrointestinal tract into the body. Assimilation is the process whereby the digested and absorbed nutrients are incorporated into bodily tissues and/or used for metabolic purposes.

The term “animal” means any animal capable of digesting lipid and protein, including avian, bovine, canine, equine, feline, hicrine, murine, ovine, and porcine animals.

The term “single package” means that the components of a kit are physically associated in or with one or more containers and considered a unit for manufacture, distribution, sale, or use. Containers include, but are not limited to, bags, boxes, bottles, shrink wrap packages, stapled or otherwise affixed components, or combinations thereof. A single package may be containers of components for measuring blood parameters or non-blood factors physically associated such that they are considered a unit for manufacture, distribution, sale, or use.

The term “virtual package” means that the components of a kit are associated by directions on one or more physical or virtual kit components instructing the user how to obtain the other components, e.g., in a bag containing one component and directions instructing the user to go to a website, contact a recorded message, view a visual message, or contact a caregiver or instructor to obtain instructions on how to use the kit.

### The Invention

In one aspect, the invention provides methods for identifying animals with a reduced ability to digest lipid. The methods comprise:
determining for the animal at least two of the factors:
   Vitamin E concentration;
   Vitamin B12 concentration;
   folate concentration;
   pancreatic lipase immunoreactivity;
   body condition score;
   skin fold thickness; and
   age
and, for the corresponding factors, determining that the animal has a reduced ability to digest lipid if:
   Vitamin E concentration is less than or equal to 5 mg/L;
   Vitamin B12 concentration is less than or equal to 200 ng/L;
   folate concentration is less than or equal to 15 μg/L;
   pancreatic lipase immunoreactivity is greater than or equal to 3.5 μg/L;
   body condition score is less than 3 on a 5 point scale;
   skin fold thickness is less than or equal to 2 mm; and
   age is equal to or greater than 70% of the average lifespan for the type of animal.

In various embodiments, the methods comprise using three, four, five, six, or seven factors, preferably three or four factors. In various preferred embodiments, the factors are Vitamin E concentration and Vitamin B12 concentration; Vitamin E concentration, Vitamin B12 concentration, and folate concentration; and Vitamin E concentration, Vitamin B12 concentration, Vitamin E concentration, and pancreatic lipase immunoreactivity. Various combinations of the factors can be selected by the skilled artisan.

In another aspect, the invention provides methods for identifying animals with a reduced ability to digest protein. The methods comprise:
determining for the animal at least two of the factors:
   Vitamin E concentration;
   Vitamin B12 concentration;
   folate concentration;
   body condition score; and
   age
and, for the corresponding factors, determining that the animal has a reduced ability to digest lipid if:
   Vitamin E concentration is less than or equal to 5 mg/L;
   Vitamin B12 concentration is less than or equal to 200 ng/L;
   folate concentration is less than or equal to 15 μg/L;
   body condition score is less than 3 on a 5 point scale; and
   age is equal to or greater than 70% of the average lifespan for the type of animal.

In various embodiments, the methods comprise using three, four, or five factors, preferably three or four factors. In various preferred embodiments, the factors are Vitamin E concentration and Vitamin B12 concentration; Vitamin E concentration, Vitamin B12 concentration, and folate concentration; and Vitamin E concentration, Vitamin B12 concentration, Vitamin E concentration, and body condition score. Various combinations of the factors can be selected by the skilled artisan.

The inventions are based upon the discovery that various blood and physical factors are predictive of an animal's ability to digest certain food ingredients, i.e., lipid and protein, particularly lipid in the form of fat.

In preferred embodiments, the animals are animals that have received their minimum daily recommended requirement of the nutritional factors, i.e., Vitamin E, Vitamin B12, and folate.

The methods are useful for identifying animals with a reduced ability to digest lipid and protein and modifying the animal's diet to ensure proper nutrition, e.g., increase lipid or protein in the diet or increase the amount of easily digestible lipid or protein in the diet. Also, the methods are useful for evaluating an animal's ability to digest lipid and protein in animals without conducting lengthy and expensive animal or clinical trials.

The methods are useful for a variety of human and non-human animals and are particularly useful for food animals such as bovines and porcines and for companion animals such as canines and felines, including dogs and cats. In some embodiments, the animal is a companion animal. A companion animal can be an animal of any species that is kept as a pet. A companion animal can also be an animal from a variety of widely domesticated species such as dogs (Canis familiaris) and cats (Felis domesticus) regardless of whether or not the animal is kept solely as a pet. Thus, companion animals include working dogs, cats kept for rodent control, and pet cats and dogs. In preferred embodiments, the animal is an animal whose age is equal to or greater than 70% of the average lifespan for the type of animal, preferably 80%, most preferably 90%.

In another aspect, the invention provides a means for communicating information about or instructions for one or more of (1) using various combinations of Vitamin E concentration; Vitamin B12 concentration; folate concentration; pancreatic lipase immunoreactivity; body condition score; skin fold thickness; and age for identifying animals with a reduced ability to digest lipid, particularly fat; (2) using various combinations of Vitamin E concentration; Vitamin B12 concentration; folate concentration; body condition score; and age for identifying animals with a reduced ability to digest protein; and (3) using the kits of the invention for identifying animals with a reduced ability to digest fat or protein. The means comprises a document, digital storage media, optical storage media, audio presentation, or visual display containing the information or instructions. Preferably, the communication means is a displayed web site, visual display, kiosk, brochure, product label, package insert, advertisement, handout, public announcement, audiotape, videotape, DVD, CD-ROM, computer readable chip, computer readable card, computer readable disk, computer memory, or combination thereof containing such information or instructions.

Useful information includes one or more of contact information for animals or their caregivers to use if they have a question about the invention and its use. The communication means is useful for instructing on the benefits of using the invention.

In another aspect, the invention provides kits suitable for identifying animals with a reduced ability to digest lipid or protein. The kits comprise in separate containers in a single package or in separate containers in a virtual package, as appropriate for the kit component, one or more of (a) instructions for identifying animals with a reduced ability to digest lipid and (b) instructions for identifying animals with a reduced ability to digest protein and at least one of (1) components required to determine Vitamin E concentration; (2) components required to determine Vitamin B12 concentration; (3) components required to determine folate concentration; (4) components required to determine pancreatic lipase immunoreactivity; (5) components required to determine body condition score; (6) components required to determine skin fold thickness; and (7) instructions for using the kit for identifying animals with a reduced ability to digest lipid or protein. When the kit comprises a virtual package, the kit is limited to instructions in a virtual environment in combination with one or more physical kit components. The kits may contain the kit instructions and components in any of various combinations as required for identifying animals with a reduced ability to digest lipid, protein, or both.

### EXAMPLES

This invention can be further illustrated by the following example, although it will be understood that the example is included merely for purposes of illustration and is not intended to limit the scope of the invention unless otherwise specifically indicated.

### Materials and Methods

### Lipid and Protein Digestibility

All participating cats were adults and in good health. A single diet was the only source of nourishment for the cats. Water was available to the cats at all times. Each cat's weight was recorded prior to the initiation of the test. Each cat was fed the amount of food required to cover its metabolizable energy requirements. The cats were fed the same diet for a pre-test period of 5 days. Each cat's weight was recorded at day 6. The fecal collection period was from day 6 through day 11. The amount of food consumed during this period was recorded. On day 6, the diet was fed together with red iron oxide as a marker, at a concentration of 1.0 g/kg of meal mass. Red marked feces were the first to be collected. Any unmarked (normal colored) feces on day 6 and 7 prior to the first appearance of the red marked feces were discarded. All red marked feces were collected, as well as all unmarked feces that were passed after the first red feces are observed. Collected feces for each cat were stored frozen at −20° C. On the morning of day 10, red iron oxide was again added to the diet (as on day 6); this time to mark the end of the trial feeding period, and the weight of each cat was recorded. Feces continue to be collected until the reappearance of the red marker. Two samples of the diet and each individual cat's fecal samples, which were first freeze dried, were sent for analyses of protein, fat, dry matter, and ash.

### Body Condition Scoring

Body condition scoring in cats was performed as follows. Each cat was placed in a standing position and visually examined from the side and from above the cat looking down at the spine. Palpation was performed on the chest to feel for the ribs, along the spine, the face, the limbs and the abdomen to assess the degree of fat accumulation. A 5-point scoring system was used, where 1 was extremely thin and 5 was extremely obese. A body condition score (BCS) of 1 was assigned to cats with visible ribs, no palpable fat, a severe abdominal tuck, and easily palpable vertebrae and wings of ilia. A BCS of 2 was assigned to cats with easily palpable ribs, minimal fat covering, obvious lumbar vertebrae, obvious waist behind the ribs, and minimal abdominal fat. A BCS of 3 was assigned to cats that were well-proportioned with a waist behind the ribs, palpable ribs with slight fat covering, and minimal abdominal fat pad. A BCS of 4 was assigned to cats that had a poorly discernible waist, moderate fat over the ribs (though still palpable), obvious rounding of the abdomen, and a moderate abdominal fat pad. A BCS of 5 was assigned to cats that had heavy fat cover over the ribs (which are not palpable), lumbar area, face and limbs; that had distention of the abdomen with no waist; and that had extensive abdominal fat deposits.

### Skin Fold Thickness

Skin fold thickness in cats was performed as follows. A patch of hair approximately 2 inches square was shaved with cordless electric clippers (close clipped, but avoiding skin burning) at a spot on the left dorsal side, just below the spine, immediately ventral to the last ribs. Mitutoyo Digimatic electronic calipers with blunted points were set to read in millimeters. A pinch of the cat's skin was taken with the fingers (no muscle or subcutaneous fat was included) and measured with the calipers (releasing with the pinching fingers to obtain the measurement). Readings from a minimum of three sites within the shaved patch were taken and the average recorded.

### Blood Components

Serum Vitamin E, Vitamin B12, folate, and pancreatic lipase immunoreactivity (PLI) were assessed on a group of cats as follows. Animals were fasted overnight prior to obtaining blood samples. A small patch of hair, large enough to visualize the jugular vein, was shaved with cordless electric clippers (close clipped, but avoiding skin burning) at a spot over either the right or left jugular vein. A 20 or 22 gauge vacutainer attached to serum separator tubes, or 20 or 22 gauge needle attached to a 3 ml or 12 ml syringe was used to obtain a minimum of 3 ml of whole blood. Blood serum was separated according to standard methodologies and frozen at −20° C. or lower until transport to appropriate laboratories for analysis.

Vitamin E analysis was performed according to HPLC (Waters Separation Module 2690). Vitamin B12 analysis was performed according to the Elecsys Vitamin B12 assay for Cobas, Application Sheet number 04745736190. Folate analysis was performed according to the Elecsys Folate II assay for Cobas, Application sheet number 03253678160. Pancreatic lipase immunoreactivity analysis was performed according to Steiner J M, Teague S R, Williams D A. Development and analytic validation of an enzyme-linked immunosorbent assay for the measurement of canine pancreatic lipase immunoreactivity in serum. Can J Vet Res. 2003; 67:175-182.

### Example 1

Seventy (70) senior cats (ages 7 to 17) were fed a standard canned food composition for 12 months and digestibility testing was conducted. Cats were continued on the same diet for their remaining lifetime. Digestibility testing was repeated every 6 months. Blood samples for serum Vitamin E, serum Vitamin B12, serum folate, and serum pancreatic lipase immunoreactivity (PLI); body condition scoring; body composition by dual energy x-ray absorptiometry (DEXA); and skin fold thickness measurements were taken on the same cats when digestibility was determined.

Correlation analysis was performed on the data between fat and protein digestibility and the other variables. To calculate correlations, analysis of covariance was used wherein fat or protein digestibility was the dependent variable, and time, cats and one of the variables were the independent variables.

For fat digestibility, the correlations were:

| Correlated with Fat | | |
|---|---|---|
| Digestibility | Correlation | P-Value |
| Vitamin B12 | 0.3600 | 0.0001 |
| PLI | −0.2754 | 0.0422 |
| Vitamin E | 0.4267 | <0.0001 |
| Skin Thickness | 0.1266 | 0.0229 |
| Body Condition Score | 0.2108 | 0.0001 |
| Age | −0.5764 | <0.0001 |
| Tissue Fat by DEXA | 0.1827 | 0.0027 |

For protein digestibility, the correlations were:

| Correlated with Protein Digestibility | Correlation | P-Value |
|---|---|---|
| Folate | 0.1762 | 0.0767 |
| Vitamin B12 | 0.3931 | 0.0002 |
| Vitamin E | 0.3595 | <0.0001 |
| Body Condition Score | 0.2243 | 0.0001 |
| Age | −0.4334 | 0.0003 |
| Tissue Fat by DEXA | 0.1217 | 0.0385 |
| Lean Body Mass | 0.2388 | 0.0069 |

Further data evaluations were performed to determine the frequency with which low fat or protein digestibility occurred with abnormal levels of the other parameters. Abnormal levels were defined as follows:

| Parameter | Abnormal levels | |
|---|---|---|
| Fat Digestibility | <80% | |
| Protein Digestibility | <77% | |
| Vitamin E | <5 | mg/L |
| Vitamin B12 | <200 | ng/L |
| Folate | <13.4 | μg/L |
| PLI | >3.5 | μg/L |
| Body Condition Score | <2.5 | |
| Skin thickness | <2 | mm |
| Age | >11 | years |

The following results were obtained for fat digestibility:

| | | | % |
|---|---|---|---|
| | | | With Low Fat |
| Parameter | Value | Unit | Digestibility |
| Vitamin E level less | 5 | mg/L | 100 |
| than or equal to | 6 | mg/L | 100 |
| | 9 | mg/L | 87 |
| Vitamin B12 level | 100 | ng/L | 92 |
| less than or equal to | 200 | ng/L | 86 |
| Folate level less than | 6 | μg/L | 75 |
| or equal to | 13.4 | μg/L | 65 |
| PLI level greater than | 3.5 | μg/L | 100 |
| or equal to | 12.9 | μg/L | 44 |
| BCS less than or | 1 | | 63 |
| equal to | 2 | | 50 |
| Skin thickness less | 1.7 | mm | 51 |
| than or equal to | 2 | mm | 49 |
| Age greater than | 11 | years | 47 |

The following results were obtained for protein digestibility:

| | | | % With Low Protein |
|---|---|---|---|
| Parameter | Value | Unit | Digestibility |
| Vitamin E level less | 5 | mg/L | 80 |
| than or equal to | 6 | mg/L | 77 |
| | 9 | mg/L | 55 |
| Vitamin B12 level | 100 | ng/L | 67 |
| less than or equal to | 200 | ng/L | 60 |
| Folate level less than | 6 | μg/L | 75 |
| or equal to | | | |
| BCS less than or | 1 | | 75 |
| equal to | 2 | | 40 |
| Age greater than | 11 | years | 34 |

The results show that various combinations of the tested factors can be used for identifying animals with a reduced ability to digest lipid and protein.

In the specification, there have been disclosed typical preferred embodiments of the invention and, although specific terms are employed, they are used in a generic and descriptive sense only and not for purposes of limitation. Obviously many modifications and variations of the invention are possible in light of the above teachings. It is therefore to be understood that the invention may be practiced otherwise than as specifically described.

As used herein and in the appended claims, the singular forms “a”, “an”, and “the” include plural reference unless the context clearly indicates otherwise. The terms “comprise”, “comprises”, and “comprising” are to be interpreted inclusively rather than exclusively.

Unless defined otherwise, all technical and scientific terms and any acronyms used herein have the same meanings as commonly understood by one of ordinary skill in the art in the field of this invention. Although any compositions, methods, kits, and means for communicating information similar or equivalent to those described herein can be used to practice this invention, the preferred compositions, methods, kits, and means for communicating information are described herein.

All references cited above are incorporated herein by reference to the extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

1. A method for identifying an animal with a reduced ability to digest lipid comprising
determining for the animal at least two of the factors:
Vitamin E concentration;
Vitamin B12 concentration;
folate concentration;
pancreatic lipase immunoreactivity;
body condition score;
skin fold thickness; and
age
and, for the corresponding factors, determining that the animal has a reduced ability to digest lipid if:
Vitamin E concentration is less than or equal to 5 mg/L;
Vitamin B12 concentration is less than or equal to 200 ng/L;
folate concentration is less than or equal to 15 μg/L;
pancreatic lipase immunoreactivity is greater than or equal to 3.5 μg/L;
body condition score is less than 3 on a 5 point scale;
skin fold thickness is less than or equal to 2 mm; and
age is equal to or greater than 70% of the average lifespan for the type of animal.

2. The method of claim 1 wherein at least three factors are determined for the animal.

3. The method of claim 1 wherein at least four factors are determined for the animal.

4. The method of claim 1 wherein the factors are Vitamin E concentration and Vitamin B12 concentration.

5. The method of claim 2 wherein the factors are Vitamin E concentration, Vitamin B12 concentration, and folate concentration.

6. The method of claim 3 wherein the factors are Vitamin E concentration, Vitamin B12 concentration, Vitamin E concentration, and pancreatic lipase immunoreactivity.

7. The method of claim 1 wherein the animal is a companion animal.

8. The method of claim 7 wherein the animal is a canine.

9. The method of claim 7 wherein the animal is a feline.

10. A method for identifying an animal with a reduced ability to digest protein comprising
determining for the animal at least two of the factors:
Vitamin E concentration;
Vitamin B12 concentration;
folate concentration;
body condition score; and
age
and, for the corresponding factors, determining that the animal has a reduced ability to digest lipid if:
Vitamin E concentration is less than or equal to 5 mg/L;
Vitamin B12 concentration is less than or equal to 200 ng/L;
folate concentration is less than or equal to 15 μg/L;
body condition score is less than 3 on a 5 point scale;
age is equal to or greater than 70% of the average lifespan for the type of animal.

11. The method of claim 10 wherein at least three factors are determined for the animal.

12. The method of claim 10 wherein at least four factors are determined for the animal.

13. The method of claim 10 wherein the factors are Vitamin E concentration and Vitamin B12 concentration.

14. The method of claim 11 wherein the factors are Vitamin E concentration, Vitamin B12 concentration, and folate concentration.

15. The method of claim 12 wherein the factors are Vitamin E concentration, Vitamin B12 concentration, Vitamin E concentration, and body condition score.

16. The method of claim 10 wherein the animal is a companion animal.

17. The method of claim 16 wherein the animal is a canine.

18. The method of claim 16 wherein the animal is a feline.

19. A means for communicating information about or instructions for one or more of (1) using various combinations of Vitamin E concentration; Vitamin B12 concentration; folate concentration; pancreatic lipase immunoreactivity; body condition score; skin fold thickness; and age for identifying animals with a reduced ability to digest lipid; (2) using various combinations of Vitamin E concentration; Vitamin B12 concentration; folate concentration; body condition score; and age for identifying animals with a reduced ability to digest protein; and (3) using the kits of the invention for identifying animals with a reduced ability to digest fat or protein, the means comprising a document, digital storage media, optical storage media, audio presentation, or visual display containing the information or instructions. Preferably, the communication means is a displayed web site, visual display, kiosk, brochure, product label, package insert, advertisement, handout, public announcement, audiotape, videotape, DVD, CD-ROM, computer readable chip, computer readable card, computer readable disk, computer memory, or combination thereof containing such information or instructions.

20. The means of claim 19 selected from the group consisting of a displayed website, brochure, product label, package insert, advertisement, displayed web site, and visual display.

21. (canceled)
